(19) United States
(12) Patent Application Publication
(10) Pub. No.: US 2004/0152777 A1
(43) Pub. Date: Aug. 5, 2004

(54) Therapeutically active compounds

(76) Inventors: William J. Wechter, Ojai, CA (US); Israel Gutierrez, Colton, CA (US); Elaine Benaksas Schwartz, Yorba Linda, CA (US); E. David Murray, Redland, CA (US)

Correspondence Address: HANA VERNY, PETERS, VERNY, JONES & SCHMITT, L.L.P., SUITE 230, 425 SHERMAN AVENUE, PALO ALTO, CA, 94306, US

(21) Appl. No.: 10/762,681

(22) Filed: Jan. 21, 2004

Related U.S. Application Data
(60) Provisional application No. 60/441,892, filed on Jan. 22, 2003.

(51) Int. Cl. A61K 31/202

(52) U.S. Cl. 514/560

(57) ABSTRACT

The present invention concerns methods for treating a disease or illness in a mammal, the method comprising administering to the mammal a composition comprising a compound in an amount effective to elicit a chemopreventative effect or a therapeutic effect or a prophylactic effect or a chemoprotective effect, the compound having the formula:

A-CH(D)-CO₂H

wherein the stereochemical configuration of the alpha carbon is predominantly R-stereoisomer or is R-stereoisomer substantially free from the S-stereoisomer and wherein A is an aliphatic moiety that is alkyl of 3 to about 30 carbon atoms, substituted alkyl of 3 to about 30 carbon atoms, alkene of 3 to about 30 carbon atoms and having from 1 to about 10 unsaturations, or substituted alkene having from 3 to about 30 carbon atoms and having from 1 to about 10 unsaturations, alkyne of 3 to about 30 carbon atoms and having from 1 to about 10 unsaturations, or substituted alkyne having from 3 to about 30 carbon atoms and having from 1 to about 10 unsaturations, and wherein D is alkyl of 1 to about 10 carbon atoms, or a physiologically acceptable ester or salt thereof.

### CROSS-REFERENCE TO RELATED APPLICATIONS

This application claims the benefit of the subject matter disclosed in prior copending Provisional Patent Application Serial No. 60/441,892 filed Jan. 22, 2003, the disclosure of which is incorporated herein by reference in its entirety.

### BACKGROUND OF THE INVENTION

This invention relates to various compounds and their use in the treatment of illness and disease.

Metabolic disorders, which include, for example, obesity and diabetes, are conditions that are prevalent in contemporary society. These two conditions may result from a variety of factors such as genetics, behavior, diet and so forth.

Diabetes is characterized by impaired glucose metabolism manifesting itself among other things by an elevated blood glucose level in the diabetic patient. Underlying defects lead to a classification of diabetes into two major groups: type 1 diabetes, or insulin dependent diabetes mellitus (IDDM), arises when patients lack insulin-producing beta-cells in their pancreatic glands. Type 2 diabetes, or non-insulin dependent diabetes mellitus (NIDDM), occurs in patients with impaired beta-cell function and alterations in insulin action.

The current treatment for type 1 diabetic patients is the injection of insulin, while the majority of type 2 diabetic patients are treated with agents that stimulate beta-cell function or with agents that enhance the tissue sensitivity of the patients towards insulin. The drugs presently used to treat type 2 diabetes include alpha-glucosidase inhibitors, insulin sensitizers, insulin secretagogues, and metformin.

Over time, almost one-half of type 2 diabetic subjects lose their response to these agents. Insulin treatment is instituted after diet, exercise, and oral medications have failed to adequately control blood glucose. The drawbacks of insulin treatment are the need for drug injection, the potential for hypoglycemia, and weight gain.

Because of the problems with current treatments, new therapies to treat type 2 diabetes are needed as well as effective new treatments for diabetes in general.

Obesity is the most common and costly nutritional problem in the U.S., affecting approximately 33% of adults. The underlying causes of obesity are complex and generally cannot be simplified to mere “overeating”. Both physiological and psychological factors may be involved.

Unfortunately, the prevalence of obesity is on the rise, health care costs directly attributed to obesity are in the tens of billions of dollars, and additional tens of billions per year are spent on weight reduction programs and special foods.

Obesity is associated with increased morbidity and mortality. It has been linked to a number of diseases including type 2 diabetes mellitus, hypertension, coronary artery disease, stroke, hypercholesterolemia, cholelithiasis, fatty liver disease, certain cancers (postmenopausal breast cancer and cancers of the colon, endometrium and kidney), musculoskeletal disorders (osteoarthritis), obstructive sleep apnea, and infertility, not to mention the social consequences and isolation that many patients with obesity experience.

Current treatment strategies have been disappointing and largely ineffective for long-term success. Certainly, every effort is made to set short-term goals and recognize the importance of lifestyle alterations in the form of increased exercise and decreased caloric intake. Drug therapy is now limited since a number of weight reduction products have been taken off the market. Surgical therapy is reserved for patients with severe obesity or those with lesser obesity who have coexisting conditions. A new treatment for obesity is a desirable goal.

Other metabolic disorders or metabolic diseases of human and animal glucose metabolism include hyperglycemia, impaired glucose tolerance and hyperinsulinemia, and insulin insensitivity or insulin resistance in general, hypertriglyceridemia, dyslipidemia, non-alcoholic fatty liver, lipodystrophy, impaired glucose tolerance, syndrome X, and so forth. Hyperglycemia is a condition where the blood glucose level is above the normal level in the fasting state, following ingestion of a meal or during a glucose tolerance test. It can occur in NIDDM as well as in obesity. Hyperglycemia can occur without a diagnosis of NIDDM. This condition is called impaired glucose tolerance or pre-diabetes. Impaired glucose tolerance occurs when the rate of metabolic clearance of glucose from the blood is less than that commonly occurring in the general population after a standard dose of glucose has been orally or parenterally administered. It can occur in NIDDM as well as obesity, pre-diabetes and gestational diabetes. Hyperinsulinemia is defined as having a blood insulin level that is above normal level in fasting state or following ingestion of a meal. It can be associated with or causative of hypertension or atherosclerosis. Insulin insensitivity, or insulin resistance, occurs when the insulin-dependent glucose clearance rate is less than that commonly occurring in the general population during diagnostic procedures.

Neurologic disorders include, for example, bipolar syndrome, Alzheimer's disease, schizophrenia, and depression, etc.

Neoplastic diseases are conditions in which abnormal proliferation of cells results in a mass of tissue called a neoplasm or tumor. Neoplasms have varying degrees of abnormalities in structure and behavior. Some neoplasms are benign while others are malignant or cancerous. An effective treatment of neoplastic disease would be considered a valuable contribution to the search for cancer preventive or curative procedures.

Cancer of the colon is common in the western world and is an important cause of morbidity and mortality, having an incidence of about 5% in the U.S. population. As with other types of cancers, cancers of the gastrointestinal tract, including colon cancer, are characterized by abnormal development in cell proliferation and differentiation in the gastrointestinal tract.

Agranulocytosis is a life-threatening condition that develops very rapidly, and that is difficult to detect even with periodic white-cell counts. The leukopenia/agranulocytosis syndrome has been described for several NSAID's, such as indomethacin, ketoprofen, and ibuprofen. Indeed, such NSAID's are contraindicated in patients whose immune systems are compromised by HIV infection, chemotherapy, ionizing irradiation, corticosteroids, immunosuppressives, etc., or by such conditions as emphysema, bronchiectasis, diabetes mellitus, leukemia, burns and the like.

Another disease for which effective treatment is needed is cystic fibrosis. Cystic fibrosis (CF) is a heritable disease that follows an autosomal recessive pattern of transmittance. It is the most common lethal genetic disease in the United States. The approximate frequency in Caucasians is 1 in 2000. Cystic fibrosis is characterized by abnormal eccrine and exocrine gland function. In particular, mucous glands produce viscous secretions that lead to chronic pulmonary disease, insufficient pancreatic and digestive function and abnormally concentrated sweat. The major source of CF morbidity is pulmonary disease.

Still another disease for which effective treatment is needed is dementia including Alzheimer's Disease (AD), which is a degenerative brain disorder associated with extensive loss of specific neuronal subpopulations and characterized clinically by progressive loss of memory, cognition, reasoning, judgment and emotional stability that gradually leads to profound mental deterioration and ultimately death. AD is a common cause of progressive mental failure (dementia) in aged humans and is believed to represent the fourth most common medical cause of death in the United States. AD has been observed in varied races and ethnic groups worldwide and presents a major present and future public health problem. The disease is currently estimated to affect up to four million individuals in the United States alone. To date, AD has proven to be incurable, and presently causes up to 50,000 deaths yearly.

A continuing need exists for compounds and compositions that are useful in the treatment of, for example, metabolic disorders including obesity and diabetes, and also for treating neoplastic disease, inflammation, cystic fibrosis, dementia, and the like. The compositions should allow for entry into tissue. The compounds and compositions should be compatible with the digestive processes of humans and other mammals.

### SUMMARY OF THE INVENTION

One embodiment of the present invention concerns a method of treating a mammal having a disease state or being pre-disposed to a disease state. The method comprises administering to a host an effective amount of pharmaceutically active compound as described below.

In one embodiment the compound employed as the pharmaceutically active compound has the formula:

A-CH(D)-CO₂H

wherein the stereochemical configuration of the alpha carbon is predominantly R-stereochemical configuration (that is, R-enantiomer) or is R stereochemical configuration substantially free from the S configuration (that is, an R-enantiomer substantially free from the S-enantiomer) and wherein A is an aliphatic moiety that is alkyl of 1 to about 30 carbon atoms, substituted alkyl of 1 to about 30 carbon atoms, alkene of 2 to about 30 carbon atoms and having from 1 to about 10 unsaturations, or substituted alkene having from 2 to about 30 carbon atoms and having from 1 to about 10 unsaturations, or alkyne having from 2 to about 30 carbon atoms and having from 1 to about 10 unsaturations, or substituted alkyne having 2 to about 30 carbon atoms and having from 1 to about 10 unsaturations, and wherein D is alkyl of 1 to about 10 carbon atoms, or a physiologically acceptable ester or salt thereof or a metabolic precursor thereof.

In one embodiment the compound employed as the pharmaceutically active compound has the formula:

A-CH(D)-CO₂H

wherein the stereochemical configuration of the alpha carbon is predominantly R-enantiomer or is R-enantiomer substantially free from S-enantiomer and wherein A is an aliphatic moiety that is alkyl of 3 to about 30 carbon atoms, substituted alkyl of 3 to about 30 carbon atoms, alkene of 3 to about 30 carbon atoms and having from 1 to about 10 unsaturations, or substituted alkene having from 3 to about 30 carbon atoms and having from 1 to about 10 unsaturations, or alkyne having from 3 to about 30 carbon atoms and having from 1 to about 10 unsaturations, or substituted alkyne having 3 to about 30 carbon atoms and having from 1 to about 10 unsaturations, and wherein D is alkyl of 1 to about 10 carbon atoms, or a physiologically acceptable ester or salt thereof or a metabolic precursor thereof.

In a particular embodiment of the compounds of the above formula, A is selected from the group consisting of CₙH₂₊₁, CₙH₂ₙ₋₁, CₙH₂ₙ₋₃, and CₙH₂ₙ₋₅ wherein n is 3 to about 20 and wherein 1 to about 5 carbon atoms are optionally substituted with alkyl, branched alkyl, alkenyl, or alkynyl groups and the like.

In one embodiment of the above A comprises a moiety of the formula:

E-(CH₂(CH₂)ₐCH(Y)(CH₂(CH₂)_{b}CH(Y))ₘ(CH₂(CH₂)_{c}—

wherein m is 0 to 3 and wherein Y are independently alkyl having from 1 to about 10 carbon atoms and wherein the carbon atoms comprising the Y groups are independently R-enantiomer substantially free from S-enantiomer, S-enantiomer substantially free from R-enantiomer, or one or more of the carbon atoms comprising the Y groups may be a combination of S-enantiomers and R-enantiomers (thereby rendering the compound wholly or partially racemic with respect to such carbon atom(s)) and a, b and c are independently an integer of 1 to 5 and wherein the carbon atoms not comprising the Y groups may be substituted with one or more substituents and wherein E is an aliphatic moiety that is alkyl of 1 to about 20 carbon atoms, substituted alkyl of 1 to about 20 carbon atoms, alkene of 2 to about 20 carbon atoms and having from 1 to about 5 unsaturations, or substituted alkene having from 2 to about 20 carbon atoms and having from 1 to about 5 unsaturations or alkyne having from 2 to about 20 carbon atoms and having from 1 to about 5 unsaturations, or substituted alkyne having 2 to about 20 carbon atoms and having from 1 to about 5 unsaturations.

In a particular embodiment of the above, the moiety has the formula:

E-CH₂CH₂CH(Y)(CH₂CH₂CH(Y))ₘCH₂CH₂CH₂— wherein E, m and Y are as described above. In another particular embodiment of the above, the moiety has the formula:

E-CH₂CH₂CH(Y)(CH₂CH₂CH₂CH₂CH(Y))ₘCH₂CH₂CH₂— wherein E, m and Y are as described above.

In a particular embodiment of the compounds of the above formulas, E is selected from the group consisting of CₙH₂ₙ₊₁, CₙH₂ₙ₋₁, CₙH₂ₙ₋₃, and CₙH₂ₙ₋₅ wherein n is 1 to about 20 and wherein 1 to about 5 carbon atoms are optionally substituted with alkyl, branched alkyl, alkenyl, or alkynyl groups and the like.

In another embodiment of the present invention, the pharmaceutically active compound has the formula:

A′-CH(D′)-CO₂H

wherein the stereochemical configuration of the alpha carbon is predominantly R-enantiomer or is R-enantiomer substantially free from S-enantiomer and wherein A′ is an aliphatic moiety that is alkyl of 5 to about 20 carbon atoms, substituted alkyl of 5 to about 20 carbon atoms, alkene of 5 to about 20 carbon atoms and having from 1 to about 5 unsaturations, or substituted alkene having from 5 to about 20 carbon atoms and having from 1 to about 5 unsaturations, or alkyne having from 5 to about 20 carbon atoms and having from 1 to about 5 unsaturations, or substituted alkyne having 5 to about 20 carbon atoms and having from 1 to about 5 unsaturations, and wherein D′ is alkyl of 1 to about 5 carbon atoms, or a physiologically acceptable ester or salt thereof or a metabolic precursor thereof.

In a particular embodiment of the compounds of the above formulas, A′ is selected from the group consisting of CₙH₂ₙ₊₁, CₙH₂ₙ₋₁, CₙH₂ₙ₋₃, and CₙH₂ₙ₋₅ wherein n is 5 to about 20 and wherein 1 to about 5 carbon atoms are optionally substituted with alkyl, branched alkyl, alkenyl, or alkynyl groups and the like.

In one embodiment of the above compounds A′ comprises a moiety of the formula:

E′-(CH₂(CH₂)ₐCH(Y)(CH₂(CH₂)_{b}CH(Y))ₘ(CH₂(CH₂)_{c}—

wherein m is 0 to 3 and wherein Y is independently alkyl having from 1 to about 5 carbon atoms and wherein the carbon atoms comprising the Y groups are independently R-enantiomer substantially free from S-enantiomer, S-enantiomer substantially free from R-enantiomer, or one or more of the carbon atoms comprising the Y groups may be a combination of S-enantiomers and R-enantiomers and a, b and c are independently an integer of 1 to 5 and wherein the carbon atoms not comprising the Y groups may be substituted with one or more substituents and

wherein E′ is an aliphatic moiety that is alkyl of 1 to about 12 carbon atoms, substituted alkyl of 1 to about 12 carbon atoms, alkene of 2 to about 12 carbon atoms and having from 1 to about 3 unsaturations, or substituted alkene having from 2 to about 12 carbon atoms and having from 1 to about 3 unsaturations or alkyne having from 2 to about 12 carbon atoms and having from 1 to about 3 unsaturations, or substituted alkyne having 2 to about 12 carbon atoms and having from 1 to about 3 unsaturations.

In a particular embodiment of the above A′ comprises a moiety of the formula: E′-CH₂CH₂CH(Y)(CH₂CH₂CH(Y))ₘCH₂CH₂CH₂—. In another particular embodiment of the above, the moiety has the formula:

E′-CH₂CH₂CH(Y)(CH₂CH₂CH₂CH₂CH(Y))ₘCH₂CH₂CH₂—.

In a particular embodiment of the compounds of the above formulas, E′ is selected from the group consisting of CₙH₂ₙ₊₁, CₙH₂ₙ₋₁, CₙH₂ₙ₋₃, and CₙH₂ₙ₋₅ wherein n is 1 to about 10 and wherein 1 to about 5 carbon atoms are optionally substituted with alkyl, branched alkyl, alkenyl, or alkynyl groups and the like.

In another embodiment of the present invention the compound is a compound wherein A′ comprises a moiety of the formula:

E′-CH₂CH₂CH(Y′)(CH₂(CH₂)_{z}CH(Y′))ₘCH₂CH₂CH₂—

wherein m is 0 to 3 and z is 1, 3, 5, 7 and so forth and wherein E′ is as described above, and wherein Y′ is independently alkyl having from 1 to about 5 carbon atoms and wherein the carbon atoms not comprising the Y′ groups may be (optionally) substituted with one or more substituents.

In one embodiment of the invention, the compounds that find use have the formula:

W-C(X)(D)-COOH, wherein X is alkyl of 1 to about 10 carbon atoms and D is alkyl of 1 to about 10 carbon atoms and wherein W is hydrogen or alkyl of 1 to about 30 carbon atoms, substituted alkyl of 1 to about 30 carbon atoms, alkene of 2 to about 30 carbon atoms and having from 1 to about 10 unsaturations, or substituted alkene having from 2 to about 30 carbon atoms and having from 1 to about 10 unsaturations, alkyne of 2 to about 30 carbon atoms and having from 1 to about 10 unsaturations, or substituted alkyne having from 2 to about 30 carbon atoms and having from 1 to about 10 unsaturations or the pharmaceutically acceptable esters thereof or a metabolic precursor thereof.

Another embodiment in accordance with the present invention includes compounds and their physiologically acceptable esters represented by the formula:

W-C(CH₃)(CH₃)CO₂H

wherein W is hydrogen or alkyl of 1 to about 20 carbon atoms, substituted alkyl of 1 to a bout 20 carbon atoms, alkene of 2 to about 20 carbon atoms and having from 1 to 2 unsaturations, or substituted alkene having from 2 to about 20 carbon atoms and having 1 to 2 unsaturations, alkyne of 2 to about 20 carbon atoms and having 1 to 2 unsaturations, or substituted alkyne having from 2 to about 20 carbon atoms and having 1 to 2 unsaturations.

Another embodiment of the present invention involves pharmaceutically acceptable esters of the compounds mentioned above.

Another embodiment of the present invention is a pharmaceutical composition comprising a compound as described above in a pharmaceutically acceptable carrier.

### BRIEF DESCRIPTION OF THE DRAWING

FIG. 1A is a schematic depicting an example of a reaction scheme for the preparation of compounds for use in accordance with the present invention.

FIG. 1B is a schematic depicting an example of a reaction scheme for the preparation of compounds for use in accordance with the present invention.

### DETAILED DESCRIPTION OF SPECIFIC EMBODIMENTS OF THE INVENTION

As mentioned above, an embodiment of the present invention is a method of treatment of a disease. The method comprises administering to a host in need thereof an amount of a compound as described herein effective in treating (as defined herein) the disease (as defined herein) or a pharmaceutically acceptable or physiologically acceptable salt thereof.

In one specific embodiment the alpha carbon of a carboxylic acid compound in accordance with the present invention contains a single hydrogen substituent rendering the alpha carbon a chiral center, in which case the stereoisomeric form of the compound that is useful in the treatment of disease is predominantly R-enantiomer or is R-enantiomer substantially free from S-enantiomer.

As mentioned above, the compound may comprise a plurality of chiral centers in a carbon chain where the chiral centers have a predetermined stereochemical configuration. On the other hand, the compound may comprise a plurality of chiral centers in a carbon chain of the compound where one or more of the chiral centers are spaced apart by a designated number of carbon atoms. The stereochemical configuration of the chiral centers that is alpha to a carboxylic acid functionality is predominantly R-enantiomer or is R-enantiomer substantially free from S-enantiomer and the stereochemical configuration of the other centers may be independently R-enantiomer substantially free from S-enantiomer, S-enantiomer substantially free from R-enantiomer or mixtures thereof.

In another specific embodiment the compound comprises a single hydrogen substituent at the alpha carbon (carbon 1 not counting the carboxyl carbon) and at carbon 5, and, alternatively, also at carbon 9, and further alternatively, also at carbon 13, and further alternatively, also at carbon 17, and further alternatively, also at carbon 21, and further alternatively, also at carbon 24, and so forth. Accordingly, the aforementioned carbon atoms of the compound represent chiral centers. In this situation, the compound may comprise R-enantiomers at each of the chiral centers. Again, not wanting to be held to any particular theory or mechanism, it is believed that the synthesis of the CoA ester in vivo resulting from the action of CoA ligase on the R-enantiomeric centers consumes much of the ATP generated during beta-oxidation. It should be pointed out that in the aforementioned embodiment S-enantiomers may be present since the S enantiomers are substantially incapable of formation of CoA esters.

In another specific embodiment the compound comprises a single hydrogen substituent at the alpha carbon (carbon 1 not counting the carboxyl carbon) and at carbon 4, and, alternatively, also at carbon 7, and further alternatively, also at carbon 10, and further alternatively, also at carbon 13, and further alternatively, also at carbon 16, and further alternatively, also at carbon 19, and so forth. Accordingly, the aforementioned carbon atoms of the compound represent chiral centers. In this situation, the compound may comprise R-enantiomers at each of the chiral centers resulting in the presence of multiple chiral centers that in this circumstance have two methylene (—CH₂—) groups between them. In this situation, after one round of beta-oxidation the compound must undergo alpha-oxidation to permit the next round of beta-oxidation. The alpha-oxidation results in a free carboxyl group as opposed to beta-oxidation, which results in a CoA ester for subsequent oxidation steps. This free carboxyl must be re-esterified to a CoA ester. It should be pointed out that in the aforementioned embodiment S-enantiomers may be present since, as explained above, the S enantiomers are substantially incapable of formation of CoA esters.

Definitions

“Aliphatic” means an open chain hydrocarbon moiety and excludes aryl, heteroaryl, and the like.

“Alkyl” means a branched or unbranched saturated monovalent hydrocarbon radical containing 1 to 30 or more carbon atoms, such as methyl, ethyl, propyl, tert-butyl, n-hexyl, iso-hexyl, n-octyl, iso-octyl, and so forth. Alkyl includes lower alkyl. “Lower alkyl” means a branched or unbranched saturated monovalent hydrocarbon radical containing 1 to 10 carbon atoms, such as methyl, ethyl, propyl, isopropyl, tert-butyl, iso-butyl, n-pentyl, iso-pentyl, and so forth. In some embodiments, the alkyl substituent is lower alkyl, such as, e.g., methyl.

“Alkene” means a branched or unbranched unsaturated hydrocarbon radical containing at least one double or ethenylic bond and 2 to 30 or more carbon atoms and includes lower alkene, unless otherwise indicated. “Lower alkene” means a branched or unbranched unsaturated hydrocarbon radical containing at least one double or ethenylic bond and 2 to 6 carbon atoms, unless otherwise indicated. One or more carbon atoms of the hydrocarbon are optionally substituted.

“Alkyne” means a branched or unbranched unsaturated hydrocarbon radical containing at least one triple or ethynylic bond and 2 to 30 or more carbon atoms and includes lower alkyne, unless otherwise indicated. “Lower alkyne” means a branched or unbranched unsaturated hydrocarbon radical containing at least one triple or ethynylic bond and 2 to 6 carbon atoms, unless otherwise indicated. One or more carbon atoms of the hydrocarbon are optionally substituted.

“Substituted” means that one or more carbon atoms of the alkyl, alkene or alkyne may comprise one or more substituents such as, for example, alkyl, alkenyl, alkynyl and the like.

“Optional” or “optionally” means that the subsequently described event or circumstance may or may not occur, and that the description includes instances where said event or circumstance occurs and instances in which it does not. For example, “optionally substituted” means that a substituent may or may not be present on the carbon atom of the hydrocarbon. For example, “optionally, other therapeutic agents” means that another therapeutic agent may or may not be present in the composition.

The term “enantiomer” as used herein refers to the stereochemical or stereoisomeric arrangement of substituents on a particular carbon atom of a molecule. Such a carbon atom is also referred to herein as a chiral center.

The phrase “predominantly an R-enantiomer” means an enantiomeric mixture that comprises at least 50% of an R-enantiomer, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, of an R-enantiomer. In general, the amount of S-enantiomer that can be tolerated depends on the toxicity of the S-enantiomer, if any, the amount of the overall mixture that can be administered conveniently to a patient, any effect that the S-enantiomer may have on the therapeutic effectiveness of the compounds of the invention, and so forth.

“Substantially free from S-enantiomer” means that the amount of S-isomer of the particular carbon atom or chiral center of the compound in question, if any, present is insufficient by itself to reduce therapeutic effectiveness or result in toxicity, if any. In some embodiments, the compound contains no more than about 1% of the corresponding S-isomer at the particular carbon atom.

The compounds of the invention include physiologically or pharmaceutically acceptable esters. “Physiologically acceptable” or “pharmaceutically acceptable” means that the ester may be consumed without deleterious effects on the consumer. Such deleterious effects include toxicity, palatability, decrease in the availability of the fat-soluble vitamins A, D, E, and K, diarrhea, loose stools, gas and abdominal cramping and the like. In some embodiments, the esters are esters of a polyhydric alcohol, which is any aliphatic or aromatic compound containing at least two free hydroxyl groups. In some embodiments the polyhydric alcohol for ester formation is glycerol, which yields triglycerides from the compounds of the invention. The polyhydric alcohol esters of the present compounds may be mono-, di- and tri-esters. In some embodiments the polyhydric alcohol esters are tri-esters.

Also included within the scope of the compounds of the present invention are pharmaceutically acceptable salts of the compounds of the present invention where the nature of the compound permits salt formation. A pharmaceutically acceptable salt refers to those salts which retain the biological effectiveness and properties of the original molecule and which are not biologically or otherwise undesirable. Such salts include, by way of example and not limitation, ammonium, potassium, sodium, and so forth.

“Metabolic precursor” means that the compound in question, when ingested, results in a compound have therapeutic effectiveness in accordance with the invention by means of metabolic processes such as, for example, oxidation processes. For example, phytol is a metabolic precursor to phytanic acid and subsequently to pristanic acid as a result of metabolic oxidation and other processes.

Compounds for Use in Invention

As mentioned above, in one embodiment the compound employed as the pharmaceutically active compound has the formula:

A-CH(D)-CO₂H

wherein the stereochemical configuration of the alpha carbon is predominantly R configuration or is R substantially free from the S configuration and wherein A is

(i) alkyl of 3 to about 30 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 5 to about 25 carbon atoms or more, about 10 to about 20 carbon atoms or more, about 15 to about 20 carbon atoms or more;

(ii) substituted alkyl of 3 to about 30 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 5 to about 25 carbon atoms or more, about 10 to about 20 carbon atoms or more, about 15 to about 20 carbon atoms or more;

(iii) alkene of 3 to about 30 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 5 to about 25 carbon atoms or more, about 10 to about 20 carbon atoms or more, about 15 to about 20 carbon atoms or more and having from 1 to about 10 unsaturations or 1 to 5 unsaturations or 1 to 3 unsaturations or 1 or 2 unsaturations, or

(iv) substituted alkene having from 3 to about 30 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 5 to about 25 carbon atoms or more, about 10 to about 20 carbon atoms or more, about 15 to about 20 carbon atoms or more and having from 1 to about 10 unsaturations, from 1 to about 5 unsaturations or 1 to 3 unsaturations or 1 or 2 unsaturations,

(v) alkyne of 3 to about 30 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 5 to about 25 carbon atoms or more, about 10 to about 20 carbon atoms or more, about 15 to about 20 carbon atoms or more and having from 1 to about 10 unsaturations, 1 to about 5 unsaturations, 1 to about 3 unsaturations, 1 to 2 unsaturations, or

(vi) substituted alkyne having from 3 to about 30 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 5 to about 25 carbon atoms or more, about 10 to about 20 carbon atoms or more, about 15 to about 20 carbon atoms or more and having from 1 to about 10 unsaturations, 1 to about 5 unsaturations, 1 to about 3 unsaturations, 1 to 2 unsaturations,

or a physiologically acceptable ester thereof or a metabolic precursor thereof. In general, the number of carbon atoms in A is dependent on the number of carbon atoms in the metabolic blocker since the overall number of carbon atoms of the hydrocarbon portion of the compound is as set forth above. Furthermore, the number of unsaturations in A is also dependent on the number of carbon atoms of the hydrocarbon portion of the compound.

In some embodiments, D is alkyl of 1 to about 5 carbon atoms, for example, D is methyl.

In a particular embodiment of the compounds of the above formula, A is selected from the group consisting of CₙH₂ₙ₊₁, CₙH₂ₙ₋₁, CₙH₂ₙ₋₃, and CₙH₂ₙ₋₅ wherein n is 3 to about 20, about 5 to about 15, about 10 to about 14 and wherein 1 to about 5 carbon atoms are optionally substituted with alkyl, branched alkyl, alkenyl, or alkynyl groups and the like.

In one embodiment of the above, A comprises a moiety of the formula:

E-(CH₂(CH₂)ₐCH(Y)(CH₂(CH₂)_{b}CH(Y))ₘ(CH₂(CH₂)_{c}—

wherein m is 0, 1, 2 or 3 and wherein Y is independently alkyl having from 1 to about 10 carbon atoms, i.e., having 1, 2, 3, 4, 5, 6, 7, 8, 9 or 10 carbon atoms, and wherein the carbon atoms comprising the Y groups are independently R-enantiomer substantially free from S-enantiomer, S-enantiomer substantially free from R-enantiomer, or one or more of the carbon atoms comprising the Y groups may be a combination of S-enantiomers and R-enantiomers and a, b and c are independently an integer of 1, 2, 3, 4 or 5, in some embodiments 1 or 3 or 5, and wherein the carbon atoms not comprising the Y groups may be substituted with one or more substituents and

wherein E is an aliphatic moiety that is

(i) alkyl of 1 to about 20 carbon atoms, usually, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19 carbon atoms and may be in the range of about 2 to about 15 carbon atoms or more, about 5 to about 12 carbon atoms or more, about 6 to about 10 carbon atoms or more;

(ii) substituted alkyl of 1 to about 20 carbon atoms, usually, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19 carbon atoms and may be in the range of about 2 to about 15 carbon atoms or more, about 5 to about 12 carbon atoms or more, about 6 to about 10 carbon atoms or more;

(iii) alkene of 2 to about 20 carbon atoms, usually, at least about 2, at least about 3, at least a bout 4, at least a bout 5, at least about 6, at least about 7, at least about 8, at least about 9, at least about 10, at least about 11, at least about 12, at least about 13, at least about 14, at least about 15, at least about 16, at least about 17, at least about 18, at least about 19 carbon atoms and may be in the range of about 3 to about 15 carbon atoms or more, about 5 to about 12 carbon atoms or more, about 6 to about 10 carbon atoms or more and having from 1 to about 10 unsaturations, from 1 to about 5 unsaturations, from 1 to about 3 unsaturations, 1 or 2 unsaturations; or

(iv) substituted alkene having from 2 to about 20 carbon atoms, usually, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19 carbon atoms and may be in the range of about 3 to about 15 carbon atoms or more, about 5 to about 12 carbon atoms or more, about 6 to about 10 carbon atoms or more or more and having from 1 to about 10 unsaturations, from 1 to about 5 unsaturations, from 1 to about 3 unsaturations, 1 or 2 unsaturations; or

(v) alkyne of 2 to about 20 carbon atoms, usually, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19 carbon atoms and may be in the range of about 3 to about 15 carbon atoms or more, about 5 to about 12 carbon atoms or more, about 6 to about 10 carbon atoms or more and having from 1 to about 10 unsaturations, from 1 to about 5 unsaturations, from 1 to about 3 unsaturations, 1 or 2 unsaturations; or

(vi) substituted alkyne having from 2 to about 20 carbon atoms, usually, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19 carbon atoms and may be in the range of about 3 to about 15 carbon atoms or more, about 5 to about 12 carbon atoms or more, about 6 to about 10 carbon atoms or more or more and having from 1 to about 10 unsaturations, from 1 to about 5 unsaturations, from 1 to about 3 unsaturations, 1 or 2 unsaturations.

In a particular embodiment of the above, the moiety has the formula:

E-CH₂CH₂CH(Y)(CH₂CH₂CH(Y))ₘCH₂CH₂CH₂— wherein E, m and Y are as described above. In another particular embodiment of the above, the moiety has the formula:

E-CH₂CH₂CH(Y)(CH₂CH₂CH₂CH₂CH(Y))ₘCH₂CH₂CH₂— wherein E, m and Y are as described above.

In a particular embodiment of the compounds of the above formulas, E is selected from the group consisting of CₙH₂ₙ₊₁, CₙH₂ₙ₋₁, CₙH₂ₙ₋₃, and CₙH₂ₙ₋₅ wherein n is 1 to about 20, 5 to about 15, 8 to about 12, and wherein 1, 2, 3, 4 or 5 carbon atoms are optionally substituted with alkyl, branched alkyl, alkenyl, or alkynyl groups and the like.

In another embodiment of the present invention, the pharmaceutically active compound has the formula:

A′-CH(D′)-CO₂H

wherein the stereochemical configuration of the alpha carbon is predominantly R configuration or is R substantially free from the S configuration and wherein A′ is an aliphatic moiety that is

(i) alkyl of 5 to about 20 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 15 carbon atoms or more, about 8 to about 12 carbon atoms or more;

(ii) substituted alkyl of 5 to about 20 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 15 carbon atoms or more, about 8 to about 12 carbon atoms or more;

(iii) alkene of 5 to about 20 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 15 carbon atoms or more, about 8 to about 12 carbon atoms or more and having from 1 to about 10 unsaturations, 1 to about 5 unsaturations, from 1 to about 3 unsaturations, 1 or 2 unsaturations, or

(iv) substituted alkene having from 5 to about 20 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 15 carbon atoms or more, about 8 to about 12 carbon atoms or more and having from 1 to about 10 unsaturations, 1 to about 5 unsaturations, from 1 to about 3 unsaturations, 1 or 2 unsaturations; or

(v) alkyne of 5 to about 20 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 15 carbon atoms or more, about 8 to about 12 carbon atoms or more and having from 1 to about 10 unsaturations, 1 to about 5 unsaturations, from 1 to about 3 unsaturations, 1 or 2 unsaturations, or

(vi) substituted alkyne having from 5 to about 20 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 15 carbon atoms or more, about 8 to about 12 carbon atoms or more and having from 1 to about 10 unsaturations, 1 to about 5 unsaturations, from 1 to about 3 unsaturations, 1 or 2 unsaturations;

or pharmaceutically acceptable esters or salts thereof or a metabolic precursor thereof.

In some embodiments, D is alkyl of 1 to about 5 carbon atoms, for example, D is methyl.

In a particular embodiment of the compounds of the above formulas, A′ is selected from the group consisting of CₙH₂ₙ₊₁, CₙH₂ₙ₋₁, CₙH₂ₙ₋₃, and CₙH₂ₙ₋₅ wherein n is 5 to about 20, 6 to about 15, 8 to about 12 and wherein 1, 2, 3, 4 or 5 carbon atoms are optionally substituted with alkyl, branched alkyl, alkenyl, or alkynyl groups and the like.

In one embodiment of the above compounds A′ comprises a moiety of the formula:

E′-(CH₂(CH₂)ₐCH(Y)(CH₂(CH₂)_{b}CH(Y))ₘ(CH₂(CH₂)_{c}—

wherein m is 0, 1, 2 or 3 and wherein Y is independently alkyl having 1, 2, 3, 4 or to about 5 carbon atoms and wherein the carbon atoms comprising the Y groups are independently R-enantiomer substantially free from S-enantiomer, S-enantiomer substantially free from R-enantiomer, or one or more of the carbon atoms comprising the Y groups may be a combination of S-enantiomers and R-enantiomers and a, b and c are independently an integer of 1, 2, 3, 4 or 5 and wherein the carbon atoms not comprising the Y groups may be substituted with one or more substituents and

wherein E′ is an aliphatic moiety that is

(i) alkyl of 1 to about 12 carbon atoms, usually, at least about 2, at least about 3, at least about 4, at least about 5, at least about 6, at least about 7, at least about 8, at least about 9, at least about 10, at least about 11 carbon atoms and may be in the range of about 2 to about 10 carbon atoms or more, about 5 to about 8 carbon atoms or more;

(ii) substituted alkyl of 1 to about 12 carbon atoms, usually, at least about 2, at least about 3, at least about 4, at least about 5, at least about 6, at least about 7, at least about 8, at least about 9, at least about 10, at least about 11 carbon atoms and may be in the range of about 2 to about 10 carbon atoms or more, about 5 to about 8 carbon atoms or more;

(iii) alkene of 2 to about 12 carbon atoms, usually, at least about 2, at least about 3, at least about 4, at least about 5, at least about 6, at least about 7, at least about 8, at least about 9, at least about 10, at least about 11 carbon atoms and may be in the range of about 2 to about 10 carbon atoms or more, about 5 to about 8 carbon atoms or more and having from 1 to about 10 unsaturations, from 1 to about 5 unsaturations, from 1 to about 3 unsaturations, 1 or 2 unsaturations; or

(iv) substituted alkene having from 2 to about 12 carbon atoms, usually, at least about 2, at least about 3, at least about 4, at least about 5, at least about 6, at least about 7, at least about 8, at least about 9, at least about 10, at least about 11 carbon atoms and may be in the range of about 2 to about 10 carbon atoms or more, about 5 to about 8 carbon atoms or more and having from 1 to about 10 unsaturations, from 1 to about 5 unsaturations, from 1 to about 3 unsaturations, 1 or 2 unsaturations; or

(v) alkyne of 2 to about 12 carbon atoms, usually, at least about 2, at least about 3, at least about 4, at least about 5, at least about 6, at least about 7, at least about 8, at least about 9, at least about 10, at least about 11 carbon atoms and may be in the range of about 2 to about 10 carbon atoms or more, about 5 to about 8 carbon atoms or more and having from 1 to about 10 unsaturations, from 1 to about 5 unsaturations, from 1 to about 3 unsaturations, 1 or 2 unsaturations; or

(vi) substituted alkyne having from 2 to about 12 carbon atoms, usually, at least about 2, at least about 3, at least about 4, at least about 5, at least about 6, at least about 7, at least about 8, at least about 9, at least about 10, at least about 11 carbon atoms and may be in the range of about 2 to about 10 carbon atoms or more, about 5 to about 8 carbon atoms or more and having from 1 to about 10 unsaturations, from 1 to about 5 unsaturations, from 1 to about 3 unsaturations, 1 or 2 unsaturations; and

wherein the number of carbon atoms in E′ is dependent on the length of the moiety other than E′. For example, when m is 1 and a, b and c are each 1, the moiety has the formula:

E′-CH₂CH₂CH(Y)CH₂CH₂CH(Y)CH₂CH₂CH₂— and E′ is alkyl of 1 to 10 carbon atoms, substituted alkyl of 1 to 10 carbon atoms, alkene of 2 to 10 carbon atoms or substituted alkene of 2 to 10 carbon atoms, or alkyne of 2 to 10 carbon atoms or substituted alkyne of 2 to 10 carbon atoms.

When m is 2 and a, b and c are each 1, the moiety has the formula:

E′-CH₂CH₂CH(Y)CH₂CH₂CH(Y)CH₂CH₂CH(Y)CH₂CH₂CH₂— wherein E′ is alkyl of 1 to 7 carbon atoms, substituted alkyl of 1 to 7 carbon atoms, alkene of 2 to 7 carbon atoms or substituted alkene of 2 to 7 carbon atoms, alkyne of 2 to 7 carbon atoms or substituted alkyne of 2 to 7 carbon atoms.

In a particular embodiment of the above, the moiety has the formula:

E′-CH₂CH₂CH(Y)(CH₂CH₂CH(Y))ₘCH₂CH₂CH₂CH₂— and E′, m and Y are as described above. In another particular embodiment of the above the moiety has the formula:

—CH₂CH₂CH(Y)(CH₂CH₂CH₂CH₂CH(Y))ₘCH₂CH₂CH₂CH₂— and E′, m and Y are as described above.

In a particular embodiment of the compounds of the above formulas, E′ is selected from the group consisting of CₙH₂ₙ₊₁, CₙH₂ₙ₋₁, CₙH₂ₙ₋₃, and CₙH₂ₙ₋₅ wherein n is 1 to about 10 and wherein 1 to about 5 carbon atoms are optionally substituted with alkyl, branched alkyl, alkenyl, or alkynyl groups and the like.

In a particular embodiment of the above A′ comprises a moiety of the formula: E′-CH₂CH₂CH(Y)(CH₂CH₂CH(Y))ₘCH₂CH₂CH₂—. In another particular embodiment of the above, the moiety has the formula:

E′-CH₂CH₂CH(Y)(CH₂CH₂CH₂CH₂CH(Y))ₘCH₂CH₂CH₂—.

In a particular embodiment of the compounds of the above formulas, E′ is selected from the group consisting of CₙH₂ₙ₊₁, CₙH₂ₙ₋₁, CₙH₂ₙ₋₃, and CₙH₂ₙ₋₅ wherein n is 1 to about 10 and wherein 1 to about 5 carbon atoms are optionally substituted with alkyl, branched alkyl, alkenyl, or alkynyl groups and the like.

In another embodiment of the present invention the compound is a compound wherein A′ comprises a moiety of the formula:

E′-CH₂CH₂CH(Y)(CH₂(CH₂)_{z}CH(Y))ₘCH₂CH₂CH₂—

wherein m is 0, 1, 2 or 3 and z is 1, 3, 5, 7 and so forth and wherein E′ is as described above, and wherein Y is independently alkyl having from 1, 2, 3, 4 or 5 carbon atoms and wherein the carbon atoms not comprising the Y groups may be (optionally) substituted with one or more substituents.

In some embodiments, Y is alkyl of 1, 2, 3, 4 or 5 carbon atoms; for example, Y is methyl.

When m is 0, the moiety has the formula:

E′-CH₂CH₂CH(Y)CH₂CH₂CH₂—.

When m is 1 and z is 1, the moiety has the formula:

E′-CH₂CH₂CH(Y)CH₂CH₂CH(Y)CH₂CH₂CH₂—.

When m is 2 and z is 1, the moiety has the formula:

E′-CH₂CH₂CH(Y)CH₂CH₂CH(Y)CH₂CH₂CH(Y)CH₂CH₂CH₂.

When m is 3 and z is 1, the moiety has the formula:

E′-CH₂CH₂CH(Y)CH₂CH₂CH(Y)CH₂CH₂CH(Y)CH₂CH₂CH(Y)CH₂CH₂CH₂—.

When m is 1 and z is 3, the moiety has the formula:

E′-CH₂CH₂CH(Y)CH₂CH₂CH₂CH₂CH(Y)CH₂CH₂CH₂—.

When m is 2 and z is 3, the moiety has the formula:

E′-CH₂CH₂CH(Y)CH₂CH₂CH₂CH₂CH(Y)CH₂CH₂CH₂CH₂CH(Y)CH₂CH₂CH₂—.

When m is 3 and z is 3, the moiety has the formula:

E′-CH₂CH₂CH(Y)CH₂CH₂CH₂CH₂CH(Y)CH₂CH₂CH₂CH₂CH(Y)CH₂CH₂CH₂CH₂—CH(Y)CH₂CH₂CH₂—.

In some embodiments, E′ is alkyl of 1 to about 10 carbon atoms, 1 to 5 carbon atoms, or 1 to 3 carbon atoms; substituted alkyl of 1 to about 10 carbon atoms, 1 to 5 carbon atoms, or 1 to 3 carbon atoms; alkene of 2 to about 10 carbon atoms, 2 to 5 carbon atoms, 2 to 3 carbon atoms and having from 1 to about 3 unsaturations, or 1 to 2 unsaturations; or substituted alkene having from 2 to about 10 carbon atoms, 2 to 5 carbon atoms, 2 to 3 carbon atoms and having from 1 to about 3 unsaturations, or 1 to 2 unsaturations; alkyne of 2 to about 10 carbon atoms, 2 to 5 carbon atoms, 2 to 3 carbon atoms and having from 1 to about 3 unsaturations, or 1 to 2 unsaturations; or substituted alkyne having from 2 to about 10 carbon atoms, 2 to 5 carbon atoms, 2 to 3 carbon atoms and having from 1 to about 3 unsaturations, or 1 to 2 unsaturations.

In a particular embodiment of the compounds of the above formulas, E′ is selected from the group consisting of CₙH₂ₙ₊₁, CₙH₂ₙ₋₁, CₙH₂ₙ₋₃, and CₙH₂ₙ₋₅ wherein n is 1 to 20, or 1 to 10, or 1 to 5 and wherein 1 to 5, or 1 to 3, carbon atoms are optionally substituted with, alkyl, alkenyl, alkynyl and the like.

The A or E (or variants thereof such as, e.g., A′ or E′ and so forth) moieties may be a “short chain alkyl” or a “long chain alkyl.” “Short chain alkyl” means an alkyl containing from about 4 to about 10 carbon atoms. “Long chain alkyl” means an alkyl containing more than 10 carbon atoms. For example, A moieties for the compounds of the aforementioned formulas, by way of illustration and not limitation, include (numbering begins with the carbon atom that is attached to the alpha carbon atom), n-butyl, n-hexyl, n-octyl, n-decyl, n-dodecyl, n-tetradecyl, n-pentadecyl, n-hexadecyl, n-heptadecyl, n-octadecyl, n-nonadecyl, n-tetradecenyl, n-hexadecenyl, 7-hexadecenyl, 11-hexadecenyl, 5-tetradecynyl, 10-hydroxy-7-hexadecyl, 7,10-hexadecadienyl, 7,10,13-hexadecatrienyl, hexadeca-9,11,13-trienyl, 10-octadecenyl, and the like.

Specific Embodiments of Compounds in Accordance with the Present Invention

One series of compounds for use in accordance with the present invention includes compounds, and their physiologically acceptable esters or salts or metabolic precursors, represented by the formula:

Z-CH(CH₃)CO₂H

wherein the alpha carbon is predominantly R-enantiomer or is R-enantiomer substantially free from S-enantiomer and Z is an aliphatic carbon chain that is

(i) alkyl of 3 to about 30 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 5 to about 25 carbon atoms or more, about 10 to about 20 carbon atoms or more, about 15 to about 20 carbon atoms or more;

(ii) substituted alkyl of 3 to about 30 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 5 to about 25 carbon atoms or more, about 10 to about 20 carbon atoms or more, about 15 to about 20 carbon atoms or more;

(iii) alkene of 3 to about 30 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 5 to about 25 carbon atoms or more, about 10 to about 20 carbon atoms or more, about 15 to about 20 carbon atoms or more and having from 1 to about 10 unsaturations, from 1 to about 5 unsaturations, from 1 to about 3 unsaturations, 1 or 2 unsaturations, or

(iv) substituted alkene having from 3 to about 30 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and maybe in the range of about 5 to about 25 carbon atoms or more, about 10 to about 20 carbon atoms or more, about 15 to about 20 carbon atoms or more and having from 1 to about 10 unsaturations, from 1 to about 5 unsaturations, from 1 to about 3 unsaturations, 1 or 2 unsaturations; or

(v) alkyne of 3 to about 30 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 5 to about 25 carbon atoms or more, about 10 to about 20 carbon atoms or more, about 15 to about 20 carbon atoms or more and having from 1 to about 10 unsaturations, from 1 to about 5 unsaturations, from 1 to about 3 unsaturations, 1 or 2 unsaturations, or

(vi) substituted alkyne having from 3 to about 30 carbon atoms, usually, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 5 to about 25 carbon atoms or more, about 10 to about 20 carbon atoms or more, about 15 to about 20 carbon atoms or more and having from 1 to about 10 unsaturations, from 1 to about 5 unsaturations, from 1 to about 3 unsaturations, 1 or 2 unsaturations.

Another series of compounds for use in accordance with the present invention includes compounds, and their physiologically acceptable esters and salts or metabolic precursors, represented by the formula:

Q-CH₂CH₂CH(CH₃)CH₂CH₂CH(CH₃)CH₂CH₂CH₂CH(CH₃)—CO₂H

wherein the carbon atom alpha to the carboxyl group is predominantly R-enantiomer or is R-enantiomer substantially free of S-enantiomer and the other carbon atoms comprising the methyl groups are independently R-enantiomer substantially free from S-enantiomer, S-enantiomer substantially free from R-enantiomer, or one or more of the other carbon atoms comprising the methyl groups may be a combination of S-enantiomers and R-enantiomers (thereby rendering the compound wholly or partially racemic with respect to such carbon atom(s)) and wherein Q is selected from the group consisting of alkyl of 1 to about 10 carbon atoms, substituted alkyl of 1 to about 10 carbon atoms, alkene of 2 to about 10 carbon atoms and having from 1 to about 5 unsaturations, 1 to about 3 unsaturations, 1 or 2 unsaturations, or substituted alkene having from 2 to about 10 carbon atoms and having from 1 to about 5 unsaturations, 1 to about 3 unsaturations, 1 or 2 unsaturations, alkyne of 2 to about 10 carbon atoms and having from 1 to about 5 unsaturations, 1 to about 3 unsaturations, 1 or 2 unsaturations, or substituted alkyne having from 2 to about 10 carbon atoms and having from 1 to about 5 unsaturations, 1 to about 3 unsaturations, 1 or 2 unsaturations. Partially racemic means that there is a preponderance of one stereoisomer over the other stereoisomer. For example, a mixture of stereoisomers may comprise at least 51%, at least 55%, at least 60%, at least 65%, at least 70%, at least 75%, at least 80%, at least 85%, at least 90%, at least 95%, at least 96%, at least 97%, at least 98%, at least 99% of one stereoisomeric or enantiomeric form over the other.

In some embodiments of the above, Q is an aliphatic moiety that is

(i) alkyl of 1 to about 10 carbon atoms, usually, at least about 2, at least about 3, at least about 4, at least about 5, at least about 6, at least about 7, at least about 8, at least about 9 carbon atoms and may be in the range of about 2 to about 9 carbon atoms or more, about 3 to about 8 carbon atoms or more, about 4 to about 7 carbon atoms or more;

(ii) substituted alkyl of 1 to about 10 carbon atoms, usually, at least about 2, at least about 3, at least about 4, at least about 5, at least about 6, at least about 7, at least about 8, at least about 9 carbon atoms and may be in the range of about 2 to about 9 carbon atoms or more, about 3 to about 8 carbon atoms or more, about 4 to about 7 carbon atoms or more;

(iii) alkene of 2 to about 10 carbon atoms, usually, at least about 2, at least about 3, at least about 4, at least about 5, at least about 6, at least about 7, at least about 8, at least about 9 carbon atoms and may be in the range of about 2 to about 9 carbon atoms or more, about 3 to about 8 carbon atoms or more, about 4 to about 7 carbon atoms or more; and having from 1 to about 5 unsaturations, from 1 to about 3 unsaturations, from about 1 to 2 unsaturations; or

(iv) substituted alkene having from 2 to about 10 carbon atoms, usually, at least about 2, at least about 3, at least about 4, at least about 5, at least about 6, at least about 7, at least about 8, at least about 9 carbon atoms and may be in the range of about 2 to about 9 carbon atoms or more, about 3 to about 8 carbon atoms or more, about 4 to about 7 carbon atoms or more; and having from 1 to about 5 unsaturations, from 1 to about 3 unsaturations, from about 1 to 2 unsaturations; or

(v) alkyne of 2 to about 10 carbon atoms, usually, at least about 2, at least about 3, at least about 4, at least about 5, at least about 6, at least about 7, at least about 8, at least about 9 carbon atoms and may be in the range of about 2 to about 9 carbon atoms or more, about 3 to about 8 carbon atoms or more, about 4 to about 7 carbon atoms or more; and having from 1 to about 5 unsaturations, from 1 to about 3 unsaturations, from about 1 to 2 unsaturations; or

(vi) substituted alkyne having from 2 to about 10 carbon atoms, usually, at least about 2, at least about 3, at least about 4, at least about 5, at least about 6, at least about 7, at least about 8, at least about 9 carbon atoms and may be in the range of about 2 to about 9 carbon atoms or more, about 3 to a bout 8 carbon atoms or more, about 4 to about 7 carbon atoms or more; and having from 1 to about 5 unsaturations, from 1 to about 3 unsaturations, from about 1 to 2 unsaturations.

Another series of compounds in accordance with the present invention includes compounds, and their physiologically acceptable esters and salts or metabolic precursors, represented by the formula:

Q′-CH₂CH₂CH₂CH₂CH(CH₃)CH₂CH₂CH₂CH₂CH(CH₃)CH₂CH₂CH₂CH(CH₃)—CO₂H

wherein the carbon atom alpha to the carboxyl group is predominantly R-enantiomer or is R-enantiomer substantially free of S-enantiomer and the other carbon atoms comprising the methyl groups are independently R-enantiomers substantially free from S-enantiomers, S-enantiomer substantially free from R-enantiomer, or one or more of the other carbon atoms comprising the methyl groups may be a combination of S-enantiomers and R-enantiomers and wherein Q′ is selected from the group consisting of alkyl of 1 to about 6 carbon atoms, substituted alkyl of 1 to about 6 carbon atoms, alkene of 2 to about 6 carbon atoms and having from 1 to about 3 unsaturations, 1 or 2 unsaturations, or substituted alkene having from 2 to about 6 carbon atoms and having from 1 to about 3 unsaturations, 1 or 2 unsaturations, alkene of 2 to about 6 carbon atoms and having from 1 to about 3 unsaturations, 1 or 2 unsaturations, or substituted alkene having from 2 to about 6 carbon atoms and having from 1 to about 3 unsaturations, 1 or 2 unsaturations, alkyne of 2 to about 6 carbon atoms and having from 1 to about 3 unsaturations, 1 or 2 unsaturations, or substituted alkyne having from 2 to about 6 carbon atoms and having from 1 to about 3 unsaturations, 1 or 2 unsaturations.

In some embodiments of the above, Q′ is an aliphatic moiety that is

(i) alkyl of 1 to about 6 carbon atoms, usually, at least about 2, at least about 3, at least about 4, at least about 5 carbon atoms and may be in the range of about 2 to about 5 carbon atoms or more, 3 to 4 carbon atoms or more;

(ii) substituted alkyl of 1 to about 6 carbon atoms, usually, at least about 2, at least about 3, at least about 4, at least about 5 carbon atoms and may be in the range of about 2 to about 5 carbon atoms or more, 3 to 4 carbon atoms or more;

(iii) alkene of 2 to about 6 carbon atoms, usually, at least about 2, at least about 3, at least about 4, at least about 5 carbon atoms and may be in the range of about 2 to about 5 carbon atoms or more, 3 to 4 carbon atoms or more; and having from 1 to about 3 unsaturations, from 1 to about 2 unsaturations; or

(iv) substituted alkene having from 2 to about 6 carbon atoms, usually, at least about 2, at least about 3, at least about 4, at least about 5 carbon atoms and may be in the range of about 2 to about 5 carbon atoms or more, 3 to 4 carbon atoms or more; and having from 1 to about 3 unsaturations, from 1 to about 2 unsaturations; or

(v) alkyne of 2 to about 6 carbon atoms, usually, at least about 2, at least about 3, at least about 4, at least about 5 carbon atoms and may be in the range of about 2 to about 5 carbon atoms or more, 3 to 4 carbon atoms or more; and having from 1 to about 3 unsaturations, from 1 to about 2 unsaturations; or

(vi) substituted alkyne having from 2 to about 6 carbon atoms, usually, at least about 2, at least about 3, at least about 4, at least about 5 carbon atoms and may be in the range of about 2 to about 5 carbon atoms or more, 3 to 4 carbon atoms or more; and having from 1 to about 3 unsaturations, from 1 to about 2 unsaturations.

One embodiment of the invention comprises methods of treating a host for a disease by administering to the host an effective amount of R,R,R-phytanic acid or R,R,R-pristanic acid or combinations thereof and including physiologically acceptable esters thereof and metabolic precursors thereof such as, for example, R,R,R-phytol as a metabolic precursor to R,R,R-phytanic acid and subsequently R,R,R-pristanic acid. Another embodiment employs R,R,S-phytanic acid or R,R,S-pristanic acid. Still other embodiments employ R,S,R-phytanic acid, S,S,R-phytanic acid, S,R,R-phytanic acid, R,S,R-pristanic acid, S,S,R-pristanic acid, S,S,R-pristanic acid, and so forth. Physiologically acceptable esters of the above compounds may be employed. In certain embodiments, metabolic precursors of the above may be employed such as, for example, R,R,R-phytol as a metabolic precursor to R,R,R-phytanic acid and subsequently R,R,R-pristanic acid, and R,R,S-phytol as a metabolic precursor to R,R,S-phytanic acid and subsequently R,R,S-pristanic acid.

Enantiomeric mixtures may also be employed (i.e., partially racemic mixtures) where the mixtures may comprise at least 51%, at least 55%, at least 60%, at least 65%, at least 70%, at least 75%, at least 80%, at least 85%, at least 90%, at least 95%, at least 96%, at least 97%, at least 98%, at least 99% of one enantiomeric form over the other for each of the chiral centers. Thus, for example, one chiral center may be one enantiomer substantially free from the other enantiomer while another chiral center may be an enantiomeric mixture and so forth.

Another embodiment of the invention includes compounds, and their physiologically acceptable esters and salts or metabolic precursors, which have the formula:

W-C(X)(D)-COOH, wherein X is alkyl of 1 to about 10 carbon atoms and D is alkyl of 1 to about 10 carbon atoms and wherein W is hydrogen or alkyl of 1 to about 30 carbon atoms, substituted alkyl of 1 to about 30 carbon atoms, alkene of 2 to about 30 carbon atoms and having from 1 to about 10 unsaturations, or substituted alkene having from 2 to about 30 carbon atoms and having from 1 to about 10 unsaturations, alkyne of 2 to about 30 carbon atoms and having from 1 to about 10 unsaturations, or substituted alkyne having from 2 to about 30 carbon atoms and having from 1 to about 10 unsaturations or the pharmaceutically acceptable esters thereof. In some embodiments, W is (i) alkyl of 1 to about 30 carbon atoms, usually, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more; (ii) substituted alkyl of 1 to about 30 carbon atoms, usually, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more; (iii) alkene of 2 to about 30 carbon atoms, usually, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more and having from 1 to about 10 unsaturations or 1 to about 5 unsaturations or 1 to about 3 unsaturations or 1 to 2 unsaturations, or (iv) substituted alkene having from 2 to about 30 carbon atoms, usually, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, a bout 10 to a bout 30 carbon atoms or more, about 15 to about 30 carbon atoms or more and having from 1 to about 10 unsaturations or 1 to about 5 unsaturations or 1 to about 3 unsaturations or 1 to 2 unsaturations; (v) alkyne of 2 to about 30 carbon atoms, usually, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more and having from 1 to about 10 unsaturations or 1 to about 5 unsaturations or 1 to about 3 unsaturations or 1 to 2 unsaturations, or (vi) substituted alkyne having from 2 to about 30 carbon atoms, usually, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of 1 to about 20 carbon atoms, 1 to about 15 carbon atoms, 1 to about 10 carbon atoms, about 6 to about 30 carbon atoms or more, about 10 to about 30 carbon atoms or more, about 15 to about 30 carbon atoms or more and having from 1 to about 10 unsaturations or 1 to about 5 unsaturations or 1 to about 3 unsaturations or 1 to 2 unsaturations.

Another embodiment in accordance with the present invention includes compounds, and their physiologically acceptable esters and salts or metabolic precursors, represented by the formula:

W′-C(CH₃)(CH₃)—CO₂H

wherein W′ is alkyl of 1 to about 20, 1 to about 10, 1 to about 5, 1 to about 4, 1 to about 3, 1 to 2, carbon atoms, substituted alkyl of 1 to about 20, 1 to about 10, 1 to about 5, 1 to about 4, 1 to about 3, 1 to 2, carbon atoms, alkene of 2 to about 20, 2 to about 10, 2 to about 5, 2 to about 4, 2 to 3, carbon atoms and having from 1 to 2 unsaturations, or substituted alkene having from 2 to about 20, 2 to about 10, 2 to about 5, 2 to about 4, 2 to 3, carbon atoms and having 1 to 2 unsaturations, alkyne of 2 to about 20, 2 to about 10, 2 to about 5, 2 to about 4, 2 to 3, carbon atoms and having 1 to 2 unsaturations, or substituted alkyne having from 2 to about 20, 2 to about 10, 2 to about 5, 2 to about 4, 2 to 3, carbon atoms and having 1 to 2 unsaturations, or a physiologically acceptable ester thereof.

Preparation of the Compounds of the Invention

The compounds of the present invention may be prepared by methods that include steps that are individually known in the art but are not known in the art in combination for the preparation of the compounds of the invention. Some of the compounds of the invention may be prepared by known polymerization techniques, which are chosen based on the particular compound of the invention being made.

Compounds that are substantially R-enantiomers: Compounds that are R-enantiomers substantially free from S-enantiomers may be prepared by the following illustrative procedures. In one approach, the starting material may be a known compound. The known compound employed as the starting material may be synthetic or natural, saturated or unsaturated, with straight or branched chains. Examples of such compounds are caproic, caprylic, pelargonic, capric, undecanoic, lauric, myristic, myristoleic, palmitic, margaric, stearic, arachidic, behenic, behenolic, erucic, erucidic (brassic), heptadecanoic, lignoceric, cerotic, montanic, mellissic, palmitoleic (zoomaric), palmitolic, ricinoleic, oleic, vaccenic, linoleic, linolenic, eleostearic, arachidonic, nervonic, eicosapentaenoic, decosatetraenoic, decosapentaenoic, decosahexaenoic, and the like acids.

The carboxyl group of the known compound is converted to a nitrile functionality. Typically, nitriles may be synthesized from the corresponding carboxylic acid by a multistep procedure beginning with the treatment of the free carboxylic acid, an ester thereof, an acid halide thereof, an anhydride thereof and so forth with a reducing agent such as, for example, a metal hydride, e.g., LiAlH₄. The specific reaction conditions will not be discussed herein. See, for example, Org. React (1951) δ 469.

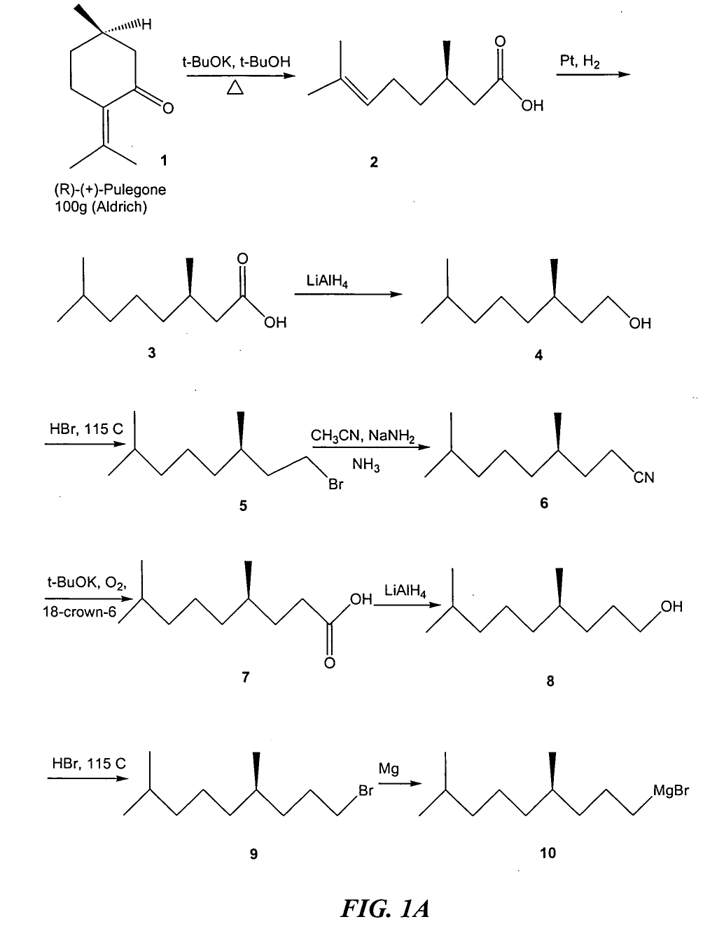

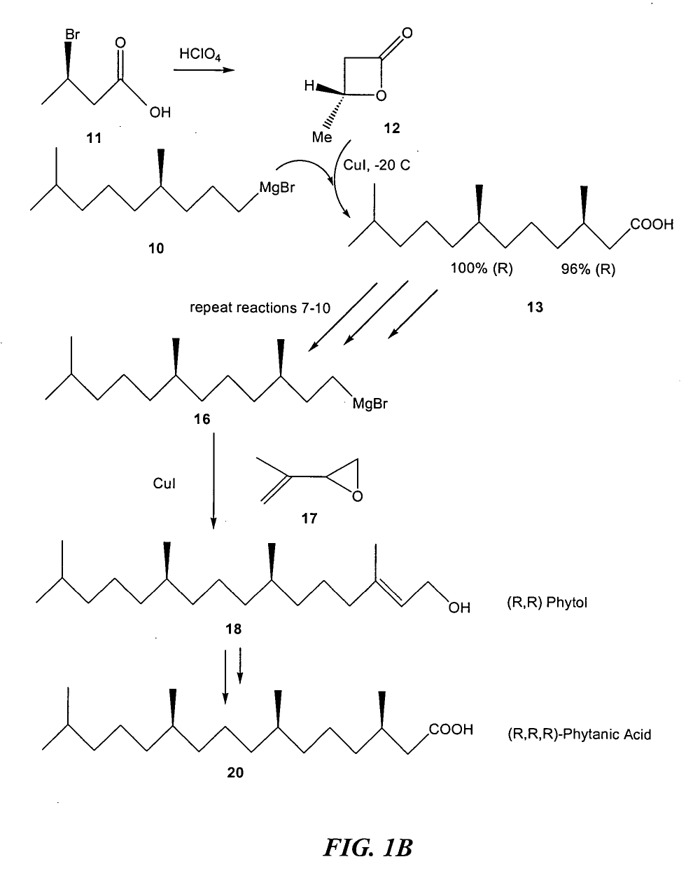

The resulting alcohol is then treated to convert the alcohol to a halide group. One approach involves reaction with hydrogen bromide at 115° C. The halogenation agent may be, for example, hydrogen chloride, hydrogen iodide, thionyl chloride and the like. A nitrile substituent is then introduced by reaction of the bromide with, for example, acetonitrile and NaNH₂ in ammonia. Then, the cyano group is hydrolyzed to give the carboxyl group. The hydrolysis may be carried out under acid or basic conditions. Usually, relatively strong acidic or basic conditions are employed. The acid may be, for example, a mineral acid such as, e.g., hydrochloric acid, sulfuric acid, phosphoric acid, and the like, an organic acid such as, e.g., trifluoroacetic acid, o-chlorobenzoic acid. In general, hydrolysis conditions are well known in the art and will not be discussed in detail. The hydrolysis is carried out in an aqueous medium usually under reflux conditions. Reduction followed by bromination and cyanation adds one carbon fragment to the molecule in question. An example of adding a chiral three-carbon fragment is illustrated in FIGS. 1A-1B. From the above derived bromide in question, a Grignard reagent is formed by reaction with magnesium. The lactone of (R)-(+)-3-bromobutyric acid is formed by reaction with perchloric acid. In the presence of cuprous iodide at −20° C., the lactone of (R)-(+)-3-bromobutyric acid is reacted with the above Grignard reagent derived from starting compound to form in very good yield, stereospecifically, the R enantiomer of the methyl substituted compound at the 3-position carbon. This same methodology can be repeated to increase chain length.

A particular example of the above procedure is depicted in FIGS. 1A-1B. (R,R,R) Phytanic Acid is the desired, physiologically relevant compound being synthesized in a stereo-controlled fashion in the procedure depicted. In FIGS. 1A-1B, t-bu is t-butyl.

Other approaches include combining a racemic mixture of the appropriate compound with a fungus and incubating the mixture under conditions for converting to one of the desired stereoisomers. For example, a racemate may be converted to R-enantiomer substantially free from S-enantiomer in this manner. The above process can be easily be performed at a larger scale by those skilled in the art.

Utility, Testing and Administration:

Utility

The above compounds and the pharmaceutically acceptable esters and salts thereof are found to possess valuable pharmacological properties. The compounds of the invention may be used in treating a disease or illness in a mammal, for example, a human. To this end, a composition comprising an enantiomerically stable form of a compound mentioned above is administered to the mammal in an amount sufficient to effect treatment of the mammal. Such treatment may include eliciting a chemoprotective effect or a therapeutic effect or a prophylactic effect.

Disease states, by way of illustration and not limitation, that are alleviated by treatment with a compound in accordance include, by way of illustration and not limitation, metabolic disorders such as, for example, obesity, diabetes and so forth, inflammation, cystic fibrosis, essential fatty acid syndrome, dementia, neoplastic disease, endocrinologic diseases and neurodegenerative diseases, and so forth. Dementia includes Alzheimer's disease, Parkinson's disease, Charcot-Marie-Tooth Disease, Amyothrophic lateral sclerosis, dementia with lewy bodies, and so forth. Inflammation includes arthritis and the like, Hepatitis and the like, inflammatory bowel disease, colitis and the like, and Crohn's disease, Systemic Lupus Erymathosis (SLE), and so forth. Neoplastic disease includes cancers and adenocarcinomas, for instance, gastrointestinal cancers including colon cancer, rectal cancer, breast cancer, ovarian, cancer, endometrial cancer, thyroid cancer, lung cancer, leukemia, lymphoma, cancer of the larynx, cervical cancer, prostate cancer, testicular cancer, bladder cancer, kidney cancer, pancreatic cancer, myeloma, squamous cell carcinoma, brain tumors, and lipoma. Dementia includes Alzheimer's disease, and so forth. Neoplastic disease includes cancers such as, for example, adenocarcinomas, for instance, gastrointestinal cancers including colon cancer, rectal cancer, breast cancer, and the like.

In addition to the above, the compounds of the present invention may have a preventative or curative effect for various diseases or illnesses. Effective to elicit a therapeutic effect means that an overall improvement in the disease state or the illness state is achieved and includes relieving the disease or illness, i.e. causing regression of the disease or illness. A therapeutically effective a mount refers to that amount which is sufficient to effect treatment, as defined above, when administered to a mammal in need of such treatment. The therapeutically effective amount will vary depending on the subject and disease state or illness state being treated, the severity of the affliction and the manner of administration, and may be determined routinely by one of ordinary skill in the art. In general, the human dose may be about 1 to 10,000 mg per 70 kilogram of body weight administered once or twice a day. Ideally, the dose is the lowest dose associated with activity and lack of other medical events.

Effective to elicit a chemoprotective effect means that abnormal cell proliferation is reduced. A method of measuring cell proliferation in animals is the Labeling Index (LI). Epithelial cells of the distal colon are stained using a histologic biomarker of proliferating cells. Microscopic examination allows for quantification of the proportion of proliferating cells in the crypts. A high proportion of proliferating cells or LI, particularly in the upper portion of the crypts, is an indicator of abnormal cell proliferation. A reduction in the LI of at least 10 to 50%, preferably at least 30% is associated with the reduction of abnormal cell proliferation. Of course, the particular compound used must be enantiomerically stable in the animal species being tested.

Chemoprevention in man and animals can also be measured by the inhibition of the conversion of the intestinal polyps, in an animal prone to polyposis, to neoplastic or cancerous legions. A min/+ mouse model can also be used to measure chemopreventive effect. Chemoprevention is achieved in this model if administration of the compound retards the spontaneous production of intestinal tumors in a min/+ mouse. Another test of chemoprotection is demonstrated by the prevention of induced tumors in a carcinogen treated mouse or rat.

Effective to elicit a prophylactic effect includes preventing the disease or illness from occurring in a subject that may be predisposed to the disease but has not yet been diagnosed as having it, inhibiting the disease or illness, i.e. arresting its development, in an early stage of disease, for example, the dysplastic stages of epithelial cancers (high PIN in prostate cancer, BRCA 1 and 2 mutations in women for the prevention of breast cancer, colon polyps in colon cancer, MRI or PET detected lesions or high A-beta proteins in the blood or CSF of patients with potential dementias. Alternatively, this treatment, which is cytostatic, usually can be used for “secondary chemoprevention” after cytoreduction therapy of a neoplastic disease by surgery or conventional cancer therapy.

With respect to diabetes, effective to elicit a prophylactic effect includes preventing the disease or illness from occurring in a subject that may be predisposed to the disease but has not yet been diagnosed as having it, inhibiting the disease or illness, i.e., arresting its development, in early stage of disease, for example, the complications of diabetes (retinopathy, neuropathy, nephropathy, dyslipidemia). Alternatively, this treatment, which is a disease modifier, usually can be used for adjuvant or secondary treatment of diabetes, prodromes and complications of diabetes or conventional diabetes therapy

For insulin resistance, effective to elicit a prophylactic effect includes preventing the disease or illness from occurring in a subject that may be predisposed to the disease but has not yet been diagnosed as having it, inhibiting the disease or illness, i.e., arresting its development, in early stage of disease, for example, insulin resistance syndromes (essential hypertension, compensatory hyperinsulinemia, coronary heart disease, diabetes, atherosclerosis, obesity, increased leptin concentrations, etc.). The presentation of insulin resistance as prodromic syndrome for disease such as: essential hypertension, compensatory hyperinsulinemia, coronary heart disease, diabetes, atherosclerosis, obesity, increased leptin concentrations etc. Alternatively, this treatment, which is a disease modifier, usually can be used for adjuvant or secondary treatment of insulin resistance syndromes, its prodromes and complications or conventional insulin resistance therapy

With regard to other metabolic disorders, effective to elicit a prophylactic effect includes preventing the disease or illness from occurring in a subject that may be predisposed to the disease but has not yet been diagnosed as having it, inhibiting the disease or illness, i.e., arresting its development, in early stage of disease, for example, metabolic disorders (hypertriglyceridemia, dyslipidemia, non-alcoholic fatty liver, lipodystrophy, impaired glucose tolerance, syndrome X, etc.). Alternatively, this treatment, which is a disease modifier, usually can be used for adjuvant or secondary treatment of metabolic disorders, its prodromes and complications or conventional metabolic disorder therapy

With regard to neurologic disorders, effective to elicit a prophylactic effect includes preventing the disease or illness from occurring in a subject that may be predisposed to the disease but has not yet been diagnosed as having it, inhibiting the disease or illness, i.e., arresting its development, in early stage of disease, for example, neurologic disorders (bipolar syndrome, Alzheimer's disease, schizophrenia, and depression, etc.) Alternatively, this treatment, which is a disease modifier, usually can be used for adjuvant or secondary treatment of neurologic disorders, its prodromes and complications or conventional neurologic disorder therapy.

Testing

Potential for a specific activity may be determined in vitro and in vivo by methods that are known in the art using the compounds of the invention such as, for example, A-beta protein ratios in blood or CSF.

Administration

The pharmaceutical compositions of the present invention comprise a compound as described above as the active ingredient and may also contain a pharmaceutically acceptable carrier, and optionally, other therapeutic ingredients. The active ingredient may be a pharmaceutically acceptable salt or ester of the compound where the nature of the compound permits salt formation.

In applying the compounds of this invention to treatment of the above condition, administration of the active compounds or salt or esters described herein can be by means of any of the accepted modes of administration for similar pharmaceutical compositions including oral, intravenous, rectal, parenteral (subcutaneous, intramuscular, intravenous), and like forms of administration, transdermal and other systemic routes of administration, and so forth. Any pharmaceutically acceptable mode of administration can be used, including solid, semi-solid or liquid dosage forms, such as, for example, tablets, troches, suppositories, pills, capsules, powders, liquids, dispersions, suspensions, solutions, elixirs, aerosols, patches and the like, preferably in unit dosage forms suitable for single administration of precise dosages, or in sustained or controlled release dosage forms for the prolonged administration of the compound at a predetermined rate.

In addition to the common forms set out above, the compounds of the present invention may also be administered by controlled release means and/or delivery devices such as those described in U.S. Pat. Nos. 3,845,770; 3,916,899; 3,536,809; 3,598,123; and 4,008,719, the disclosures of which are hereby incorporated by reference in their entireties.

The amount of active compound administered, of course, will be dependent on the active compound itself, the subject being treated, the severity of the affliction, the manner of administration and the judgment of the prescribing physician. In general, the amount of the compound administered is that which is sufficient to bring about the desired therapeutic effect or a chemoprotective effect or a prophylactic effect and the like. An effective dosage is usually in the range of from about 1.0 mg to about 10,000 mg per day in one or more doses. In one approach, the composition may be administered in an amount of from about 5 mg to about 5,000 mg once or twice a day. In one approach, the composition may be administered in an amount of from about 10 mg to about 2,000 mg once or twice a day. In one approach, the composition may be administered in an amount of from about 20 mg to about 1,000 mg once or twice a day. Pharmaceutical compositions of the present invention suitable for oral administration may be presented as discrete units such as capsules, cachets, or tablets, or aerosol sprays, each containing a predetermined amount of the active ingredient, as a powder or granules, or as a solution or a suspension in an aqueous liquid, a non-aqueous liquid, an oil-in-water emulsion, or a water-in-oil liquid emulsion. Such compositions may be prepared by any of the conventional methods of pharmacy, but all methods include the step of bringing into association the active ingredient with the carrier, which constitutes one or more necessary ingredients.

The compositions will typically include a conventional pharmaceutical carrier or excipient and an active compound of the invention or the pharmaceutically acceptable salts or esters thereof and, in addition, may include other medicinal agents, pharmaceutical agents, carriers, adjuvants, etc. Carriers such as starches, sugars, microcrystalline cellulose, diluents, granulating agents, lubricants, binders, disintegrating agents, and the like may be used in the cases of oral solid preparations. Oral solid preparations (such as powders, capsules, and tablets) are preferred over oral liquid preparations. In certain embodiments, oral solid preparations are gel capsules for those compounds of the invention that are oils at room temperature. Tablets may also be employed and may be coated by standard aqueous or non-aqueous techniques.

In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent. In one embodiment, each tablet contains from about 1 mg to about 10,000 mg of the active ingredient, and each cachet or capsule contains from about 1 mg to about 10,000 mg of the active ingredient. In one embodiment, the tablet, cachet or capsule may contain dosages such as, for example, about 1 mg, about 50 mg, about 100 mg, about 200 mg, about 500 mg, about 1000 mg, about 2000 mg, and so forth, of the active ingredient.

For solid compositions, conventional non-toxic solid carriers include, for example, pharmaceutical grades of mannitol, lactose, starch, magnesium stearate, sodium saccharin, talcum, cellulose, sodium crosscarmellose, glucose, sucrose, magnesium carbonate, and the like may be used. The active compound as defined above may be formulated as suppositories using, for example, polyalkylene glycols, for example, propylene glycol, as the carrier. Liquid pharmaceutically administratable compositions can, for example, be prepared by dissolving, dispersing, etc. an active compound as defined above and optional pharmaceutical adjuvants in a carrier, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 16th Edition, 1980. The composition or formulation to be administered will, in any event, contain a quantity of the active compound(s) in an amount effective to alleviate the symptoms of the subject being treated.

Dosage forms or compositions containing active ingredient in the range of 0.025 to 95% with the balance made up from non-toxic carrier may be prepared.

For oral administration, a pharmaceutically acceptable non-toxic composition is formed by the incorporation of any of the normally employed excipients, such as, for example pharmaceutical grades of mannitol, lactose, starch, magnesium stearate, sodium saccharin, talcum, cellulose, sodium crosscarmellose, glucose, sucrose, magnesium, carbonate, and the like. Such compositions take the form of solutions, suspensions, tablets, capsules, powders, sustained release formulations and the like. Such compositions may contain about 0.1% to about 95%, 0.2% to about 80%, 0.5% to about 70%, 0.5% to about 60%, 1% to about 50%, 1% to about 40%, 1% to about 30%, and so forth of active ingredient.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, etc.

Another approach for parenteral administration employs the implantation of a slow-release or sustained-release system, such that a constant level of dosage is maintained. See, e.g., U.S. Pat. No. 3,710,795, which is hereby incorporated by reference.

The percentage of active compound contained in such parental compositions is highly dependent on the specific nature thereof, as well as the activity of the compound and the needs of the subject. However, percentages of active ingredient of 0.01% to 10% in solution are employable, and will be higher if the composition is a solid, which will be subsequently diluted to the above percentages. In one embodiment, the composition may comprise about 0.01% to about 50%, about 0.01% to about 40%, about 0.01% to about 30%, about 0.01% to about 20%, about 0.01% to about 10%, about 0.02% to about 8%, of the active agent in solution.

For systemic administration via suppository, traditional binders and carriers include, e.g. polyalkylene glycols or triglycerides. Such suppositories may be formed from mixtures containing active ingredient in the range of about 0.01% to about 50%, about 0.01% to about 40%, about 0.01% to about 30%, about 0.01% to about 20%, about 0.01% to about 10%, about 0.01% to about 8%, about 0.01% to about 2%, of the active agent.

In order to aid in patient compliance with daily dosage requirements, the compounds may also be administered by formulating them in toothpaste. The compound is dissolved in a suitable solvent such as, e.g., an ethyl alcohol solution and added to the toothpaste so that the final concentration of the active ingredient is from about 0.01 to about 10%, 0.05% to about 8%, 0.1% to about 5%, 0.5% to about 1%, on a weight compositions of the present invention basis.

The compositions of the present invention may also be formulated for administration in any convenient way by analogy with other topical compositions adapted for use in mammals. These compositions may be presented for use in any conventional manner with the aid of any of a wide variety of pharmaceutical carriers or vehicles. For such topical administration, a pharmaceutically acceptable non-toxic formulation can take the form of semisolid, liquid, or solid, such as, for example, gels, creams, lotions, solutions, suspensions, ointments, powders, or the like. As an example, the active components may be formulated into a gel using ethanol, propylene glycol, propylene carbonate, polyethylene glycols, diisopropyl adipate, glycerol, water, etc., with appropriate gelling agents, such as Carbomers, Klucels, etc. If desired, the formulation may also contain minor amounts of non-toxic auxiliary substances such as preservatives, antioxidants, pH buffering agents, surface active agents, and the like. Actual methods of preparing such dosage forms are known, or will be apparent, to those skilled in the art; for example, see Remington's Pharmaceutical Sciences, Mack Publishing Company, Easton, Pa., 16th Edition, 1980.

Specific Utilities

The magnitude of a prophylactic or therapeutic dose of a compound as described above in the acute or chronic management of cancer or neoplastic disease will vary with the particular compound, the severity of the condition to be treated, and the route of administration. The dose and/or the dose frequency also vary according to the age, body weight, and response of the individual patient.

In general and as mentioned above, the total daily dose range for a compound of the invention, for the conditions described herein, is from about 1 mg to about 10,000 mg per 70 kilogram of body weight, in single or divided doses.

In managing a patient, the therapy should be initiated at a lower dose, perhaps about 1 mg to about 1000 mg and increased depending on the patient's global response. It is further recommended that infants, children, patients over 65 years, and those with impaired renal or hepatic function, initially receive low doses, and that they be titrated based on individual response(s) and blood level(s).

It may be necessary to use dosages outside these ranges in some cases as will be apparent to those skilled in the art. Further, it is noted that the ordinary skilled clinician or treating physician will know how and when to interrupt, adjust or terminate therapy in consideration of individual patient response.

The present method of treatment of colorectal cancer will be enhanced by the use of a compound as described above as an adjuvant to known chemotherapeutic agents such as 5-fluorouracil and the like.

In accordance with the present invention, cystic fibrosis patients are treated with a compound that is at an effective cystic fibrosis therapeutic amount. As used herein, an “effective cystic fibrosis therapeutic amount” is an amount that relieves CF symptoms, which can be measured by improved pulmonary function. The amount is preferably administered in a divided dose based on the plasma half-life of the particular compound.

Additionally, administration of a compound as described above appears to prevent or delay the onset of Alzheimer's Disease. Thus, in accordance with the present invention, patients at risk of developing Alzheimer's Disease are treated with a compound as described above at high dose, that is, at an effective Alzheimer's Disease prophylactic amount. As used herein, an “effective Alzheimer's Disease prophylactic amount” is that amount which will delay the onset of symptoms of AD by at least 6 months. The amount administered is usually in a divided dose based on the plasma half-life of the particular compound.

The compounds of the invention may be administered as part of a food composition, for example, in conjunction with, or in replacement of, a fat component of the food composition. The amount of compound of the invention replacing a fat component may be about 0.1 to about 99.9%, about 1 to about 99%, about 2 to about 98%, about 3 to about 97%, about 4 to about 96%, about 5 to about 95%, about 10 to about 90%, about 20 to about 80%, about 30 to about 70%, about 40 to about 60%, about 50 to about 50%, and so forth.

In addition to food compositions, the compounds of the present invention can also be used in formulating lubricants, skin creams, pharmaceutical ointments, and the like.

The invention is further illustrated by reference to the following examples describing the preparation of some compositions comprising compounds of the present invention. It will be apparent to those skilled in the art that many modifications, both to materials and methods, may be practiced without departing from the purpose and interest of this invention.

It should be understood that the above description is intended to illustrate and not limit the scope of the invention. Other aspects, advantages and modifications within the scope of the invention will be apparent to those skilled in the art to which the invention pertains. The following examples are put forth so as to provide those of ordinary skill in the art with examples of how to make and use the methods and products of the invention, and are not intended to limit the scope of what the inventors regard as their invention.

### EXAMPLE 1

### Composition for Oral Administration

| The composition contains: | % wt./wt. |
|---|---|
| Active ingredient | 20% |
| Lactose | 79.5% |
| Magnesium stearate | 0.5% |

The two ingredients are mixed and dispensed into capsules containing 500 mg each.

### EXAMPLE 2

### Composition for Oral Administration

| The composition contains: | % wt./wt. |
|---|---|
| Active ingredient | 20.0% |
| Gel capsule material | 80.0% |

The capsule is formed containing 500 mg of active ingredient.

### EXAMPLE 3

### Suppository Formulation

| The composition contains: | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

### EXAMPLE 4

Topical Formulation

| Ingredients | grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. 100 |

All of the above ingredients, except water, are combined and heated to 60° C. with stirring. A sufficient quantity of water at 60° C. is then added with vigorous stirring to emulsify the ingredients, and water then added q.s. 100 g.

In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs.

All publications and patent applications cited in this specification are herein incorporated by reference as if each individual publication or patent application were specifically and individually indicated to be incorporated by reference, except insofar as they may conflict with those of the present application (in which case the present application prevails). Methods recited herein may be carried out in any order of the recited events that is logically possible, as well as the recited order of events.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method for treating a disease or illness in a mammal, said method comprising administering to said mammal a composition comprising a compound in an amount effective to elicit a chemopreventative effect or a therapeutic effect or a prophylactic effect or a chemoprotective effect, said compound having the formula:
A-CH(D)-CO₂H
wherein the stereochemical configuration of the alpha carbon is predominantly R-stereoisomer or is R-stereoisomer substantially free from the S-stereoisomer and wherein A is an aliphatic moiety that is alkyl of 3 to about 30 carbon atoms, substituted alkyl of 3 to about 30 carbon atoms, alkene of 3 to about 30 carbon atoms and having from 1 to about 10 unsaturations, or substituted alkene having from 3 to about 30 carbon atoms and having from 1 to about 10 unsaturations, alkyne of 3 to about 30 carbon atoms and having from 1 to about 10 unsaturations, or substituted alkyne having from 3 to about 30 carbon atoms and having from 1 to about 10 unsaturations, and wherein D is alkyl of 1 to about 10 carbon atoms, or a physiologically acceptable ester or salt thereof or a metabolic precursor thereof.

2. A method according to claim 1 wherein said disease or illness is diabetes, obesity, inflammation, cystic fibrosis, dementia, or neoplastic disease.

3. A method according to claim 1 wherein said mammal is a human.

4. A method according to claim 1 wherein said composition is administered orally, transdermally, intravenously or by suppository.

5. A method according to claim 1 wherein said composition is administered in an amount of from about 1.0 mg to about 10,000 mg per day in one or more doses.

6. A method according to claim 1 wherein said composition is administered in an amount of from about 10 mg to about 2000 mg once or twice a day.

7. A method according to claim 1 wherein said composition comprises a pharmaceutically acceptable carrier.

8. A method according to claim 1 wherein A is selected from the group consisting of CₙH₂ₙ₊₁, CₙH₂ₙ₋₁, CₙH₂ₙ₋₃, and CₙH₂ₙ₋₅ wherein n is 3 to about 20 and wherein 1 to about 5 carbon atoms are optionally substituted with alkyl, branched alkyl, alkenyl, or alkynyl groups.

9. A method according to claim 1 wherein A comprises a moiety of the formula:
E-(CH₂(CH₂)ₐCH(Y)(CH₂(CH₂)_{b}CH(Y))ₘ(CH₂(CH₂)_{c}—
wherein m is 0 to 3 and wherein Y are independently alkyl having from 1 to about 10 carbon atoms and wherein the carbon atoms comprising the Y groups are independently R-enantiomer substantially free from S-enantiomer, S-enantiomer substantially free from R-enantiomer, or one or more of the carbon atoms comprising the Y groups may be a combination of S-enantiomers and R-enantiomers and a, b and c are independently an integer of 1 to 5 and wherein the carbon atoms not comprising the Y groups may be substituted with one or more substituents and
wherein E is an aliphatic moiety that is alkyl of 1 to about 20 carbon atoms, substituted alkyl of 1 to about 20 carbon atoms, alkene of 2 to about 20 carbon atoms and having from 1 to about 10 unsaturations, or substituted alkene having from 2 to about 20 carbon atoms and having from 1 to about 10 unsaturations, alkyne of 2 to about 20 carbon atoms and having from 1 to about 10 unsaturations, or substituted alkyne having from 2 to about 20 carbon atoms and having from 1 to about 10 unsaturations.

10. A method according to claim 10 wherein A comprises a moiety of the formula:
E-CH₂CH₂CH(Y)(CH₂CH₂CH(Y))ₘCH₂CH₂CH₂—.

11. A method according to claim 10 wherein A comprises a moiety of the formula:
E-CH₂CH₂CH(Y)(CH₂CH₂CH₂CH₂CH(Y))ₘCH₂CH₂CH₂—.

12. A method according to claim 1 wherein said compound is R,R,R-phytanic acid or R,R,R-pristanic acid or a physiologically acceptable salt thereof or R,R,R-phytol as a metabolic precursor to R,R,R-phytanic acid and R,R,R-pristanic acid.

13. A method for treating a disease or illness in a mammal, said method comprising administering to said mammal a composition comprising a compound in an amount effective to elicit a chemopreventative effect or a therapeutic effect or a prophylactic effect or a chemoprotective effect, said compound having the formula:
A′-CH(D′)-CO₂H
wherein the stereochemical configuration of the alpha carbon is predominantly R configuration or is R substantially free from the S configuration and wherein A′ is an aliphatic moiety that is alkyl of 5 to about 20 carbon atoms, substituted alkyl of 5 to about 20 carbon atoms, alkene of 5 to about 20 carbon atoms and having from 1 to about 10 unsaturations, or substituted alkene having from 5 to about 20 carbon atoms and having from 1 to about 5 unsaturations, alkyne of 5 to about 20 carbon atoms and having from 1 to about 10 unsaturations, or substituted alkyne having from 5 to about 20 carbon atoms and having from 1 to about 5 unsaturations, and wherein D′ is alkyl of 1 to about 5 carbon atoms,
or a physiologically acceptable ester or salt thereof or a metabolic precursor thereof.

14. A method according to claim 13 wherein said disease or illness is a metabolic disorder, inflammation, cystic fibrosis, dementia, or neoplastic disease.

15. A method according to claim 13 wherein said mammal is a human.

16. A method according to claim 13 wherein said composition is administered orally, transdermally, intravenously or by suppository.

17. A method according to claim 13 wherein said composition is administered in an amount of from about 1.0 mg to about 10,000 mg per day in one or more doses.

18. A method according to claim 13 wherein said composition is administered in an amount of from about 10 mg to about 2000 mg once or twice a day.

19. A method according to claim 13 wherein said composition comprises a pharmaceutically acceptable carrier.

20. A method according to claim 13 wherein A′ is selected from the group consisting of CₙH₂ₙ₊₁, CₙH₂ₙ₋₁, CₙH₂ₙ₋₃, and CₙH₂ₙ₋₅ wherein n is 5 to about 20 and wherein 1 to about 5 carbon atoms are optionally substituted with alkyl, branched alkyl, alkenyl, or alkynyl groups.

21. A method according to claim 13 wherein A′ comprises a moiety of the formula:
E′-(CH₂(CH₂)ₐCH(Y)(CH₂(CH₂)_{b}CH(Y))ₘ(CH₂(CH₂)_{c}—
wherein m is 0 to 3 and wherein Y is independently alkyl having from 1 to about 5 carbon atoms and wherein the carbon atoms comprising the Y groups are independently R-enantiomer substantially free from S-enantiomer, S-enantiomer substantially free from R-enantiomer, or one or more of the carbon atoms comprising the Y groups may be a combination of S-enantiomers and R-enantiomers and a, b and c are independently an integer of 1 to 5 and wherein the carbon atoms not comprising the Y groups may be substituted with one or more substituents and
wherein E′ is an aliphatic moiety that is alkyl of 1 to about 12 carbon atoms, substituted alkyl of 1 to about 12 carbon atoms, alkene of 2 to about 12 carbon atoms and having from 1 to about 5 unsaturations, or substituted alkene having from 2 to about 12 carbon atoms and having from 1 to about 5 unsaturations, alkyne of 2 to about 12 carbon atoms and having from 1 to about 5 unsaturations, or substituted alkyne having from 2 to about 12 carbon atoms and having from 1 to about 5 unsaturations.

22. A method according to claim 21 wherein A′ comprises a moiety of the formula:
E′-CH₂CH₂CH(Y)(CH₂CH₂CH(Y))ₘCH₂CH₂CH₂—.

23. A method according to claim 21 wherein A′ comprises a moiety of the formula:
E′-CH₂CH₂CH(Y)(CH₂CH₂CH₂CH₂CH(Y))ₘCH₂CH₂CH₂—.

24. A method according to claim 21 wherein

25. A method according to claim 21 wherein E′ is selected from the group consisting of CₙH₂ₙ₊₁, CₙH₂ₙ₋₁, CₙH₂ₙ₋₃, and CₙH₂ₙ₋₅ wherein n is 1 to about 10 and wherein 1 to about 5 carbon atoms are optionally substituted with alkyl, branched alkyl, alkenyl, or alkynyl groups.

26. A method according to claim 22 wherein E′ is selected from the group consisting of CₙH₂ₙ₊₁, CₙH₂ₙ₋₁, CₙH₂ₙ₋₃, and CₙH₂ₙ₋₅ wherein n is 1 to about 10 and wherein 1 to about 5 carbon atoms are optionally substituted with alkyl, branched alkyl, alkenyl, or alkynyl groups and the like.

27. A method according to claim 21 wherein A′ comprises a moiety of the formula:
E′-CH₂CH₂CH(Y)(CH₂(CH₂)_{z}CH(Y))ₘCH₂CH₂CH₂—
wherein m is 0 to 3 and z is 1, 3, 5, or 7 and wherein Y is independently alkyl having from 1 to about 5 carbon atoms and wherein the carbon atoms not comprising the Y groups are optionally substituted with one or more substituents.

28. A method according to claim 13 wherein the compound has the formula:
Z-CH(CH₃)—CO₂H
wherein the alpha carbon is predominantly R-enantiomer or is R-enantiomer substantially free from S-enantiomer and Z is an aliphatic carbon chain that is alkyl of 3 to about 10 carbon atoms, substituted alkyl of 3 to about 10 carbon atoms, alkene of 3 to about 10 carbon atoms having 1 to 2 unsaturations, or substituted alkene having from 3 to about 10 carbon atoms having 1 to 2 unsaturations, alkyne of 3 to about 10 carbon atoms having 1 to 2 unsaturations, or substituted alkyne having from 3 to about 10 carbon atoms having 1 to 2 unsaturations.

29. A method according to claim 13 wherein the compound has the formula:
Q-CH₂CH₂CH(CH₃)CH₂CH₂CH(CH₃)CH₂CH₂CH₂CH(CH₃)—CO₂H
wherein the carbon atom alpha to the carboxyl group is predominantly R-enantiomer or is R-enantiomer substantially free of S-enantiomer and the other 30 carbon atoms comprising the methyl groups are independently R-enantiomers substantially free from S-enantiomers, S-enantiomers substantially free from R-enantiomers or mixtures thereof, and wherein Q is selected from the group consisting of alkyl of 1 to about 10 carbon atoms, substituted alkyl of 1 to about 10 carbon atoms, alkene of 2 to about 10 carbon atoms and having from 1 to about 3 unsaturations, or substituted alkene having from 2 to about 10 carbon atoms and having from 1 to about 3 unsaturations, alkyne of 2 to about 10 carbon atoms and having from 1 to about 3 unsaturations, or substituted alkyne having from 2 to about 10 carbon atoms and having from 1 to about 3 unsaturations.

30. A method according to claim 29 wherein the compound has the formula:
Q′-CH₂CH₂CH₂CH₂CH(CH₃)CH₂CH₂CH₂CH₂CH(CH₃)CH₂CH₂CH₂CH(CH₃)—CO₂H
wherein the carbon atom alpha to the carboxyl group is predominantly R-enantiomer or is R-enantiomer substantially free of S-enantiomer and the other carbon atoms comprising the methyl groups are independently R-enantiomers substantially free from S-enantiomers, S-enantiomers substantially free from R-enantiomers or mixtures thereof, and wherein Q′ is selected from the group consisting of alkyl of 1 to about 6 carbon atoms, substituted alkyl of 1 to about 6 carbon atoms, alkene of 2 to about 6 carbon atoms and having from 1 to 2 unsaturations, or substituted alkene having from 2 to about 6 carbon atoms and having from 1 to 2 unsaturations, alkyne of 2 to about 6 carbon atoms and having from 1 to 2 unsaturations, or substituted alkyne having from 2 to about 6 carbon atoms and having from 1 to 2 unsaturations.

31. A method for treating a disease or illness in a mammal, said method comprising administering to said mammal a composition comprising an enantiomerically stable form of a compound in an amount effective to elicit a chemopreventative effect or a therapeutic effect or a prophylactic effect or a chemoprotective effect, said compound having the formula:
W-C(X)(D)-COOH
wherein X is alkyl of 1 to about 10 carbon atoms and D is alkyl of 1 to about 10 carbon atoms and wherein W is hydrogen or alkyl of 1 to about 30 carbon atoms, substituted alkyl of 1 to about 30 carbon atoms, alkene of 2 to about 30 carbon atoms and having from 1 to about 10 unsaturations, or substituted alkene having from 2 to about 30 carbon atoms and having from 1 to about 10 unsaturations, alkyne of 2 to about 30 carbon atoms and having from 1 to about 10 unsaturations, or substituted alkyne having from 2 to about 30 carbon atoms and having from 1 to about 10 unsaturations or the pharmaceutically acceptable esters thereof or a metabolic precursor thereof.

32. A method according to claim 31 wherein X is methyl, D is methyl and W is hydrogen or alkyl of 1 to about 20 carbon atoms, substituted alkyl of 1 to about 20 carbon atoms, alkene of 2 to about 20 carbon atoms and having from 1 to 2 unsaturations, or substituted alkene having from 2 to about 20 carbon atoms and having 1 to 2 unsaturations, alkyne of 2 to about 20 carbon atoms and having 1 to 2 unsaturations, or substituted alkyne having from 2 to about 20 carbon atoms and having 1 to 2 unsaturations, or a physiologically acceptable ester thereof.

33. A method according to claim 32 wherein W is hydrogen.
